(19) United States
(12) Patent Application Publication
(10) Pub. No.: US 2004/0110247 A1
(43) Pub. Date: Jun. 10, 2004

(54) New molecular tools for the rapid assessment of the presence and viability of microorganisms and methods of use

(76) Inventors: Philippe Moreillon, Lausanne (CH); Yok Ai Que, Lausanne (CH); Steve Aellen, Lausanne (CH)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/637,958

(22) Filed: Aug. 8, 2003

Related U.S. Application Data
(60) Provisional application No. 60/402,015, filed on Aug. 8, 2002.

(51) Int. Cl. C12Q 1/68; C12Q 1/18

(52) U.S. Cl. 435/32; 435/6

(57) ABSTRACT

Overall ratios of ribosomal DNA and ribosomal RNA in microorganisms following exposure to an antimicrobial is shown to correspond to the presence and viability of the microorganism. Methods are provided to assess the presence and viability of microorganisms, by administering an antimicrobial to a population of microorganisms having a first and second marker, quantifying the first and second markers, and determining the ratio between the quantity of the first and second marker. A concordant result indicates the presence of viable microorganisms, whereas a discordant result indicates the presence of non-viable microorganisms.

### RELATED APPLICATIONS

This application claims priority to U.S. S. No. 60/402,015, filed on Aug. 8, 2002, which is incorporated herein by reference in its entirety.

### FIELD OF THE INVENTION

The present invention relates generally to the detection and identification of microorganisms. In particular, the invention relates to the detection of the presence of microorganisms and to assessing its viability using the same molecular system. More particularly, the invention relates to the rapid detection and viability of microorganisms, in vitro or in vivo, using real-time quantitative PCR. Methods of use encompass diagnostic assay procedures, as well as methods of treating, and monitoring treatment efficacy in, a patient with a microbial infection.

### BACKGROUND OF THE INVENTION

Microorganisms flourish under many conditions and are often found in food, in drinking water, and in physiological fluid specimens including, blood, urine, spinal fluid, and the like. The attachment of microorganisms to solid surfaces is also a well known phenomenon. The ease with which microorganisms accumulate at surfaces or colonize host tissues is the cause of numerous economic and biological problems. For instance, microorganisms will readily colonize man-made structures immersed in aqueous environments which can lead to corrosion and fouling. Moreover, many diseases of animals and plants result from the colonization, followed by the growth of pathogenic microorganisms and their dissemination on or into host tissues. The attachment of bacteria to food surfaces, including meat, contributes to food spoilage and the risk of food poisoning. For example, Listeria monocytogenes is an important food borne pathogen which may contaminate meat, cheese and other foodstuffs. The attachment of L. monocytogenes to solid surfaces including glass, stainless steel, polypropylene and rubber surfaces has also been reported. Thus, it is desirable to be able to rapidly screen food, water, and other comestibles, for contamination by a pathogen.

The detection of microorganisms in patient samples is similarly necessary in the treatment of numerous infectious diseases. In the latter case, it is frequently desirable to be able to specifically type the microorganism, and would be further desirable to determine its ability to start a new infection center, and/or screen the microorganism for sensitivity to various antimicrobials.

While some microbial infections are readily treatable by administering antibiotics or some other bactericidal or bacteriostatic agent, tolerance or resistance to such treatment is problematic and can result in failure of the treatment and sometimes in death. Nosocomial pathogens can be particularly tolerant or resistant to treatment and can often result from the most common procedures, such as use of an indwelling catheter or mechanical ventilation, or from more drastic procedures, such as various surgical procedures.

Surgical wounds often penetrate far into the body. Colonization and/or infection of such a wound thus poses a grave risk to the patient. S. aureus is one of the most important causative agents of infections in surgical wounds. S. aureus is unusually adept at colonizing and invading surgical wounds; sutured wounds can be infected by far fewer S. aureus cells then are necessary to cause infection in normal skin. Invasion, or even colonization without local signs of infection of a surgical wound can lead to severe S. aureus septicaemia. Invasion of the blood stream by S. aureus can lead to seeding and infection of internal organs, particularly heart valves and bone, causing systemic diseases, such as endocarditis and osteomyelitis.

The detection and identification of microorganisms have traditionally been accomplished by pure culture isolation and identification procedures that make use of knowledge of specimen source, growth requirements, visible (colony) growth features, microscopic morphology, staining reactions, and biochemical characteristics. However, these procedures do not indicate whether the pathogen is still viable. As used herein, the term “viable” refers to the ability of a pathogen to carry out those biochemical and genetic processes, including gene expression (i.e., transcription), and DNA and RNA replication, that allow it to colonize, replicate, and propagate under suitable conditions. For purposes of the instant specification, pathogens that require the presence of a host cell in order to propagate are considered to be “viable” so long as they are capable of colonization, infection, replication, and propagation in the presence of a suitable environment. Moreover, in the case of pathogenic bacteria, viability usually connotes infectivity or attachment. Thus, the instant methods may be used to detect bacterial pathogens that remain infectious.

Assessment of microbial death and viability relies on time-consuming biological tests. Classically, microorganisms taken from drug-exposed cultures or clinical samples are innoculated on agar or liquid growth media, and growth of the surviving organisms is monitored. This process may take days to weeks for the most common bacteria and for Mycobacterium tuberculosis, and even up to one year for Mycobacterium leprae. Moreover, some poorly-cultivable or non-cultivable microorganisms may never grow in vitro due to particular, and as yet undetermined, growth requirements. Examples include Bartonella spp., Tropheryma whipplei, and Coxiella spp., which are increasingly implicated in severe infections that are difficult to manage or control. Furthermore, in order to insure that the prescribed antimicrobial or antibacterial agent is in fact effective, repeated tests during treatment are required.

In addition to microbial examination of clinical samples such as body fluids, it is often necessary to rapidly analyze the microbial content of other specimens, such as water, food, and pharmaceutical products. For example, in cooling water systems; e.g., as used in cooling towers, it is necessary to determine bacterial content in order to ascertain appropriate decontamination and treatment, as with an appropriate biocide. Furthermore, disabled bacteria, such as those debilitated by cooking or partial heat sterilization, are a major detection problem in many food processing situations. Such disabled bacteria frequently remain viable, and thus potentially pathogenic, yet are sufficiently weakened so that detection by conventional assay protocols may require a non-selective recovery step (pre-enrichment) followed by a selective enrichment step to allow growth of the targeted bacteria while growth of competing organisms is inhibited. Such additional steps can significantly add to the time required to perform the assay.

The need for simple, rapid assessment of the presence and viability of pathogens that can be performed via the same system, in vitro or in vivo, is thus clear.

### SUMMARY OF THE INVENTION

The present invention provides methods for rapidly assessing the presence of viable microorganisms in various kinds of samples. This invention is not limited to particular microorganisms and can be extended at least to prokaryotes (including, but not limited to bacteria), fungi, viruses and parasites. It also allows the monitoring of drug-induced killing of microorganisms and provides methods for determining the efficacy of drug treatment for infections, including those induced by non-cultivable pathogens.

Specifically, the invention includes methods for assessing viability of pathogenic cells having a first marker and a second marker, quantifying the first and second markers, and determining the ratio between the quantity of the first and second markers. A positive-positive ratio indicates that cells are present and viable, whereas a positive-negative ratio indicates that cells were present but non-viable.

As used herein, the terms “positive-positive ratio” or “positive-negative ratio” refer to the comparison between the quantity of the first and second markers where the first marker tracks the presence of the bacteria, irrespective of their viability, and the second marker tracks the viability of the microorganism. The first marker can be a stable molecule of the bacterial organism that is present even after death of the organism. Such molecules can include, for example, chromosomal or plasmid DNA. Typically, these molecules persist for a prolonged period of time, i.e., at least up to 50 hours following administration of an antimicrobial. For example, the first marker may be a housekeeping gene whose expression is constant in time, regardless of the state of the microorganism.

The second marker is preferably an unstable molecule of the organism, the production of which requires energy expenditure by the organism. Thus, such an unstable molecule is only present in live microorganisms. Such molecules can include, but are not limited to, mRNA or ribosomal RNA. The half-life of RNA is in the fento-seconds range in bacteria. Thus, if the microorganism is no longer viable, then transcription of the gene into RNA, which in turn is incorporated into the ribosome, will no longer occur. At this stage, it is of great importance to select a marker whose presence is constant throughout the life cycle of the microorganism. For example, it may be a housekeeping gene whose expression is constant in time, regardless of the state of the live microorganism. More particularly, it can include the ribosomal 16S or 23S genes, whose transcription is constant over the whole cell cycle. The quantification of the first and second markers can be performed by quantitative real-time PCR, preceded by reverse transcription of the second marker.

The invention also includes methods of detecting the presence or absence of microorganisms in a test sample by determining the presence or absence of a stable marker persisting for a prolonged period of time in the test sample, for example following administration of an antibiotic or other such antimicrobial, and quantifying the stable marker, if present. The presence of the stable marker following administration of the antimicrobial to the test sample, indicates that microorganisms were once present in the test sample. The absence of the stable marker in the test sample indicates the converse however.

The test sample can include, but is not limited to, (i) a mammalian tissue or secretion, e.g., blood, urine, or cerebral liquor, or (ii) a fluid or environmental sample, e.g., from a drinking source. The sample can be provided in vitro. The test sample can also be provided from food, pharmaceuticals, or any other material suitable for microbial growth. Furthermore, the invention also includes specific methods to determine the viability of bacteria once their presence has been previously established.

(1) The determination of the efficacy of a treatment for a microbial infection can be monitored by administering the treatment to a subject having a microbial infection requiring such treatment, obtaining a sample from the subject, quantifying a first and second marker in the sample following administration of the treatment, and determining the ratio between the quantity of the first and second markers. A positive-positive ratio indicates that the microorganisms are present and viable despite administration of the treatment. Thus, that particular course of treatment lacks efficacy in treating the microbial infection. Conversely, a positive-negative ratio indicates that, although the microorganisms are present, they are non-viable following administration of the treatment. Thus, that particular course of treatment is efficacious in treating the microbial infection. The treatment can include administration of an antibiotic, or other suitable antimicrobial, to the subject, which can be a mammal, e.g., a human. The antimicrobial administered can include, but is not limited to, beta-lactams (penicillin, ampicillin, piperacillin, imipenem), quinolones (levofloxacin, ciprofloxacin, norfloxacin, moxifloxacin), chloramphenicol, aminoglycosides (gentamicin, amikacin) glycopeptides (vancomycin, teioplanin), or antifungals (fluconazole, voriconazole, amphotericin B). Those skilled in the art will recognize that other antibiotics or antimicrobials, alone or in combination, can be administered to perform the methods of the present invention.

(2) The invention also includes methods for assessing antimicrobial tolerance or resistance of a population of microorganisms by administering an antimicrobial to the population of cells, which have a first and second marker, quantifying the first and second markers following administration of the antimicrobial, and determining the ratio between the quantity of the first and second markers. A positive-positive ratio indicates resistance or tolerance of the microorganisms to the particular antimicrobial, whereas a positive-negative ratio indicates susceptibility of the microorganisms to the antimicrobial-induced killing.

(3) Another aspect of the invention includes methods for diagnosing a microbial infection in a patient by obtaining at least one sample from the patient and detecting the presence or absence of microorganisms in the sample by determining the presence or absence of a stable marker persisting for a prolonged period of time in the test sample following administration of an antibiotic or other suitable antimicrobial, and quantifying the stable marker, if present. The presence of the stable marker following administration of the antibiotic or other suitable antimicrobial to the test sample, indicates that microorganisms are present in the test sample and that the patient has a microbial infection. The absence of the stable marker in the test sample indicates the converse however.

(4) The invention further includes methods of selecting a treatment for a patient with a microbial infection by obtaining from the patient at least one sample containing microorganisms having a first and second marker, administering an antimicrobial to the sample in vitro, quantifying the first and second marker following administration of the antimicrobial, determining the ratio between the quantity of the first and second marker, wherein a positive-positive ratio indicates that the microorganisms are resistant or tolerant to the antimicrobial, and wherein a positive-negative ratio indicates that the microorganisms are susceptible to the antimicrobial-induced killing, and selecting the antimicrobial for continued administration to the patient, provided that the ratio between the first and second marker is positive-negative, or repeating the procedure with an alternative antimicrobial if the ratio between the first and second marker for the first, or immediately preceding, antimicrobial is positive-positive.

(5) The invention additionally includes methods of monitoring treatment efficacy in a patient having a microbial infection by obtaining serial samples from a patient undergoing treatment for a microbial infection, quantifying the first and second markers in the sample, determining the ratio between the quantity of the first and second markers, and comparing the ratios determined at each time point. The development or maintenance of a positive-positive ratio over time indicates that the microorganisms continue to be, or have become, resistant or tolerant to the antimicrobial, whereas the development or maintenance of a positive-negative ratio over time indicates that the microorganisms continue to be, or have become, susceptible to the antimicrobial-induced killing.

(6) The invention also includes methods of screening at least one candidate compound for efficacy against antimicrobial resistant microorganisms, such as those responsible for nosocomial infections, by exposing the at least one candidate compound to the resistant microorganisms, which has a first and a second marker, quantifying the first and second markers, and determining the ratio between the quantity of the first and second markers. A positive-positive ratio indicates that the at least one candidate compound is not effective against the antimicrobial resistant microorganisms. Conversely, a positive-negative ratio indicates that the at least one candidate compound is effective against the antimicrobial resistant microorganisms. The same procedure can also be used to screen for compounds effective against antimicrobial tolerant microorganisms. In that procedure, a positive-positive ratio indicates that the at least one candidate compound is not effective against the tolerant bacteria, whereas a positive-negative ratio indicates that the at least one candidate compound is effective against the tolerant bacteria. For example, the candidate compound can include, but is not limited to, penicillin, levofloxacin, chloramphenicol, ciprofloxacin, or any other antibiotic, antimicrobial, or therapeutic compounds, alone or in combinations thereof, that inhibit bacterial growth.

(7) A further aspect of the invention includes methods for the detection of infectious agents used as biological weapons. Rapid and conclusive analytical tools are an important element in government and public health efforts to detect, deter, and contain the preparation and use of such agents. While a number of methods are currently being developed for this purpose, they all tend to lack one or more of the following critical performance factors: sensitivity, specificity, reproducibility, speed. The present invention affords the development of assays that overcome some of the shortcomings of other methods by combining the advantages of infectivity assays (showing that the agent is viable and thus capable of growth, and therefore is likely to be infectious) and those of PCR assays (showing that the agent is specifically and conclusively what it purports to be, and with high sensitivity), with the advantage of relative assay speed. The combinations of infectivity and PCR methods provides an optimal combination of specificity, sensitivity, and speed.

Samples of interest for testing in such analytical detection methods include, but are not limited to: specimens derived from potential weapons, weapons delivery devices and storage containers (suitable for delivering or carrying such biological substances, or ordinarily used by those skilled in the arts), specimens derived from production and/or purification vessels and formulation devices ordinarily used by those skilled in the art, specimens derived from cell bank containers or inoculum generation containers, specimens derived from environments potentially contaminated with suspected biological weapons, and specimens derived from humans or animals potentially contaminated with suspected biological weapons.

All technical and scientific terms used herein have the same meanings commonly understood by one of ordinary skill in the art to which this invention belongs, unless otherwise indicated. Although any methods and materials similar or equivalent to those described herein can be used to practice the present invention, the preferred methods and materials are now described. The citation or identification of any reference within this application shall not be construed as an admission that such reference is available as prior art to the present invention. All publications mentioned herein are incorporated by reference herein in their entirety.

### DETAILED DESCRIPTION OF THE INVENTION

The present invention involves methods for assessing drug-induced killing and treatment response. Although other assessment methods have previously been considered, (See, e.g., Loeliger et al. (2003), “Antibiotic-Dependent Correlation Between Drug Induced Killing and Loss of Luminescence in S. gordonni Expressing Luciferase”, Microbial Drug Resistance 9(2):123-131, the entire contents of which are hereby incorporated by reference herein), this concept is based on the comparison between an unvarying and constant, i.e., stable, marker that reflects the presence or absence of bacterial pathogens, and an unstable viability marker whose presence provides information on the capacity of the bacterial pathogen to proliferate and start new infection centers. Thus, a single system allows for double detection of two separate markers, which correspond to the presence of microorganisms and to their viability, respectively.

The markers are components of the microorganisms. For example, the stable molecular marker can include chromosomal or plasmid DNA molecules. Preferably the stable marker is located on a chromosome and is a housekeeping gene, i.e., a gene that is expressed throughout the cell cycle, because it encodes proteins required for basic functioning. Thus, the chromosomally derived marker will be constant in time regardless of the state of the microorganism, i.e., whether the microorganism is alive or dead. The unstable molecular marker can include, for example, mRNA or ribosomal RNA. These molecules are naturally unstable in live microbial cells, i.e., the half-life of mRNA or rRNA is in the fentoseconds range, largely due to the action of RNAse. Since cellular energy is required to produce this type of molecule, i.e., to transcribe DNA into RNA, non-viable cells will contain little or none of these molecules.

The 16S rRNA component of the ribosome complex is an example of a preferred marker, because it is derived from a housekeeping gene. One of ordinary skill in the art would recognize that other housekeeping genes, such as tRNA, other rRNAs or ribosomal proteins, and RNA polymerase subunits, may be substituted. 16S has a gene located on a bacterial chromosome. Thus, the 16S DNA is transcribed into RNA, which is then incorporated into the 30S subunit of the ribosome complex. Nucleotide sequencing of the 16S marker also allows for identification of the bacterial cell at the species level because different bacteria vary in their 16S ribosomal RNA sequences.

Following administration of an antimicrobial to a population of cells having a first and second marker, the markers are quantitated via real-time Polymerase Chain Reacion (PCR), or qPCR. Polymerase chain reaction (PCR) is a powerful nucleic amplification technique that can be used for the detection of bacterial pathogens whose in vitro cultivation is difficult or lengthy, or as a substitute for other methods which require the presence of living specimens for detection. In its simplest form, PCR is an in vitro method for the enzymatic synthesis of target polynucleotides, using two oligonucleotide primers that hybridize to opposite strands and flank the region of interest in the target polynucleotide. A repetitive series of cycles involving template denaturation, primer annealing, and the extension of the annealed primers by DNA polymerase results in the exponential accumulation of a specific fragment whose termini are defined by the 5′ ends of the primers. PCR reportedly is capable of producing a selective enrichment of a specific DNA sequence by a factor of 10¹². The PCR method is described in Saiki et al., 1985, Science 230:1350.

qPCR refers to a PCR reaction performed in such a way and under such controlled conditions that the results of the assay are quantitative, i.e., the assay is capable of quantifying the amount of target polynucleotide present in the sample. For the ribosomal RNA (unstable marker) the qPCR is preceded by reverse transcription of the rRNA. This process is called Reverse Transcriptase PCR (RT-PCR). RT-PCR is a variant of the basic PCR method wherein the starting material is an RNA, which is reverse transcribed into a cDNA prior to PCR (see U.S. Pat. No. 5,262,311). “RT-qPCR” refers to RT-PCR performed under conditions that afford quantitation of the RNA present in the sample.

Quantitation of the specific target polynucleotide is accomplished by performing PCR under appropriate conditions and measuring, either directly or indirectly, the production of amplified copies of the target polynucleotide. An especially useful method to quantify the target polynucleotide is by use of the TaqMan®. assay (PE Biosystems, Foster City, Calif.; see also U.S. Pat. No. 5,210,015). However, any method that allows detection and quantitation of amplified products that are produced as a result of performing the polymerase chain reaction on a specific polynucleotide target is suitable for use in the instant assay. Examples of other such methods include, but are not limited to, quantitative competitive PCR, or PCR followed by gel electrophoresis with direct quantitation of the amplicon band in the gel by densitometry.

After quantitating the ribosomal DNA and ribosomal RNA, a ratio between the overall bacterial mass and the population of viable bacteria is provided. A concordant ratio, i.e., a positive-positive ratio, indicates the presence of bacteria that remain viable following administration of an antibiotic, anti-microbial, therapeutic compounds, or combinations thereof, capable of inhibiting bacterial growth. Conversely, a discordant ratio, i.e., a positive-negative ratio, indicates that bacteria were present but are now non-viable, i.e., non-infectious, following the administration of an antibiotic or other suitable inhibitor of bacterial growth.

Such methods are particularly useful for determining the presence and viability of bacteria that are difficult to cultivate in vitro. For example, disabled bacteria, such as those debilitated by cooking or partial heat sterilization, are a major detection problem in many food processing situations. Such disabled bacteria frequently remain viable (and thus potentially pathogenic) yet are sufficiently weakened so that detection by conventional assay protocols may require a non-selective recovery step (pre-enrichment) followed by a selective enrichment step to allow growth of the targeted bacteria while growth of competing organisms is inhibited. Such additional steps can significantly add to the time required to perform the assay and result in a decreased sensitivity of the assay. The methods of the present invention however, are well suited to detect the presence and viability of such disabled bacteria by tracking the quantity of the bacteria's ribosomal DNA and RNA, following administration of the antibiotic, or other suitable inhibitor of bacterial growth, alone or in combination thereof.

In addition to assessing the presence or absence as well as the viability of microorganisms, the methods of the invention can be used to determine the efficacy of treatment for a microbial infection that is non-cultivable in vitro. By administering an antimicrobial to a subject, including human subjects, having a microbial infection requiring such treatment and obtaining a sample from said subject, the stable and unstable molecular markers of the microorganisms present in the sample can be quantified using qPCR and RT-qQPCR, respectively. A ratio of the microbial mass to the population of viable microorganisms can be obtained, thereby providing information on the efficacy of treatment. For example, a concordant ratio indicates lack of efficacy of the antimicrobial in treating the infection, because the microorganisms are both present in the sample and viable following treatment. A discordant ratio indicates that the treatment is efficacious because, although the microorganisms were present in the sample, they are no longer viable and have been killed by the antimicrobial.

The methods disclosed herein can be used to determine whether a particular microorganism is either resistant, tolerant, or susceptible to the antimicrobial. Antibiotic tolerance is a particular trait that allows the microorganism to escape the bactericidal effect of beta-lactams and other antibiotics. Tolerance is not synonymous with resistance however. While tolerant microorganisms are immune of antibiotic-induced killing, they remain fully susceptible to growth inhibition by the drug. On the other hand, antibiotic-resistant microorganisms are able to grow in spite of the presence of relatively large concentrations of the antimicrobial. However, above this new, increased minimal inhibitory concentration (MIC), they remain sensitive to drug-induced killing.

Thus, tolerance and resistance represent two different phenotypes acquired by bacteria in response to the antibiotic selective pressure operating in the clinical environment. Both are problematic because they may result in antibiotic treatment failure against a number of bacterial pathogens. Moreover, tolerance and resistance rely on genetically and mechanistically independent features that must be solved in order to better understand and prevent the ongoing escalade of antibiotic resistance.

The methods of the invention can be used to assess antibiotic resistance, tolerance, or susceptibility in a population of microorganisms. For example, by administering an antimicrobial to a population of microorganisms, which have a first and second molecular marker, the first and second marker can be quantified to determine the overall amount of microbial mass to the amount of viable microorganisms. A concordant ratio is indicative of resistance or tolerance to the antimicrobial because the microorganisms are present in the sample and viable. A discordant result is indicative of microorganisms that are susceptible to the antimicrobial because microorganisms were present in the sample, but were killed, and are now non-viable, in the presence of the antimicrobial.

A major problem with diagnosis and treatment of microbial infections is the frequent lack of correlation between a patient's symptomatic response to antimicrobial treatment and successful treatment. In order to successfully treat a disease caused by microorganisms, the rapid and accurate detection and identification of the disease-causing microorganism is required. For example, the duration of treatment for infective endocarditis remains unsolved particularly when blood cultures test sterile and pathological examination of the surgically resected cardiac valve reveals the presence of bacteria. Once the bacteria are discovered, the question arises as to whether they are still viable and thus able to continue proliferating and to start a new infection center.

The principles encompassed by the invention can be extended to diagnosing whether a patient has an on-going microbial infection, such as, but not limited to, endocarditis, or determining whether bacteria have successfully colonized on some other surface or material. For example, by obtaining at least one sample, e.g., a physiological fluid or tissue sample from a patient, such as a mammal, including humans; or a sample from food or a drinking source, etc., determining the presence or absence of a stable molecular marker that persists for a prolonged period of time in the test sample following administration of an antimicrobial agent, and quantifying the stable marker if present, it can be determined whether a patient or some other source is breeding bacteria. The presence of the stable marker indicates the presence of microorganisms in the test sample, which further indicates that the patient or other source has a microbial infection that could need treatment. Any suitable biological sample derived from the examined subject, including, but not limited to, blood, plasma, blood cells, saliva or cells derived by mouth wash, bronchial lavage or throat/skin swabs, and any body secretions such as urine and tears, liquor, etc, may be used.

Following diagnosis, the methods of the invention can select and/or optimize treatment for a patient with a microbial infection. For instance, at least one sample can be obtained from the patient having a microbial infection. The methods for assessing antimicrobial resistance, tolerance, or susceptibility can be performed to determine if the antimicrobial tested will be effective in treating the patient. If a concordant ratio results, then the microorganisms in the sample are both present and viable following administration of the first antimicrobial, which indicates resistance or tolerance. If the resulting ratio is discordant, then the microorganisms in the sample are present but non-viable, indicating antimicrobial susceptibility. Such an antimicrobial may prove to be suitable for administration for treatment. Continuing administration of the antimicrobial providing the discordant result can treat the patient for the microbial infection. However, if a concordant ratio is obtained then the procedure should be repeated with an alternative antimicrobial until an alternative antimicrobial tested yields a discordant ratio.

It is generally known that microorganisms become resistant to drugs through evolution. Resistance to an anti-infective agent develops in microorganisms during the course of patient anti-infective therapy. Through mutational events at the molecular level, microorganisms modify the molecular structures of their proteins, most commonly enzymes that regulate growth or metabolism. Mutations are normal, and occur in the absence of anti-infective therapy, but mutations in proteins that are targets for anti-viral, anti-bacterial, and anti-fungal therapeutic agents can modify the affinities between the target and the agent, or prevent interaction or access to the target's active sites, thereby nullifying the agent's ability to deliver a therapeutic effect and destroy the microorganism. Drug therapy exerts a selection pressure on the microorganisms that selects for mutations that allow the microorganism to survive, resulting in re-infection of the patient with microbe displaying a new drug-resistant phenotype.

For example, spontaneous tolerant mutants of S. gordonni have emerged during serial exposures to high concentrations of penicillin. Such a method is less stringent than gene insertion-activation shotgun mutagenesis using transposons or suicide plasmids. Rather, it allows the bacterium to “choose” its preferential, natural modifications that will most likely represent the ones encountered in the clinical situation. Several spontaneous mutants were obtained and their metabolic and genetic modifications were studied (See Caldelari et al., (2000) AAC, 44:2802-2810). Using a proteomic approach, it was observed that several spontaneous tolerant mutants over-expressed one or two protein spots that represented the products of the arc (arginine deaminase) operon. Deregulation of the arc operon in these mutants was studied by inserting a luciferase-reporter gene in this very locus, and measuring light emission during bacterial growth. In the penicillin-killed parent, luminescence was low during exponential growth and abruptly increased during the post-exponential and early stationary phases. In contrast, luminescence was constitutively expressed during the whole growth curve in the several tolerant mutants including Tol1, thus confirming the over-expression of the arc operon.

The genetic link between the tolerance mutation and the deregulation of arc was further analyzed in Tol1. Genetic crosses and DNA sequencing indicated the following: (i) transformation of the tol1 tolerance mutation into the penicillin-killed parent strain was always associated with a deregulation of the arc operon, as expressed by a luciferase reporter system; (ii) while the tol1 mutation always increased arc expression, it was physically remote, i.e., genetically unlinked, from the arc operon; (iii) the putative tol1 mutation could be located on a large (200 kb) chromosomal fragment; (iv) the two genes over-expressed in the Tol1 mutant (adi [arginine deaminase] and oct [ornithine carbamyl transferase]) were not directly involved in the tolerance phenotype; (v) their nucleotide sequence and their regulatory regions were identical on the parent and Tol1 mutant; (vi) the arc regulatory region contained a DNA stretch homologous to a catabolite repression element (CRE), suggesting that arc might be regulated in the frame of some nutritional stress response. Taken together, these observations indicate that spontaneously acquired tolerance is associated with mutations resulting in the specific deregulation of the arc operon. Moreover, the alteration appears to be common since the same was observed in ≧60% of independent mutants.

That arc is regulated by catabolite repression was confirmed in a series of experiments using chemically defined media depleted or supplemented with a number of different nutrients. Although depletion in essential amino acids or vitamins blocked bacterial growth, it did not affect arc expression. In contrast, depletion of glucose was associated with arc induction and glucose supplementation with arc repression in wild type S. gordonii. In the tolerant mutant Tol1, on the other hand, glucose depletion or supplementation did not affect arc expression. This indicates that Tol1 is indeed directly or indirectly affected in its catabolite repression regulation.

Once antibiotic resistant bacteria are discovered, the methods of the invention can be used to screen at least one candidate compound for efficacy against the resistant bacteria. There are now a number of bacterial species which increasingly exhibit resistance to one or more classes of anti-microbial agents, making it that much more important to perform susceptibility testing. Failure of a particular susceptibility test to accurately predict antimicrobial resistance in a patient's isolate could significantly impact patient care if an antibiotic is used to which the microorganism is not susceptible. Different types of susceptibility tests can be used to test a microorganism. The following brief descriptions give details of some known susceptibility tests as well as some details that relate to the present invention.

One type of susceptibility test is the disk diffusion test, often referred to as the Kirby-Bauer test. This is a standardized test that involves inoculating (with 0.5 McFarland standardized suspension of a microbial isolate) a gel plate (e.g. a 150-mm Mueller-Hinton agar plate) and placing thereon one or more disks impregnated with fixed concentrations of antibiotics. After incubation (e.g. 18-24 hours at 35 degrees C.), the diameter of zones of inhibition around the disks (if present) determine the sensitivity of the inoculated microorganism to the particular antimicrobial agent impregnated in each disk. Due to the standardization of the Kirby-Bauer method, results of this method are analyzed by comparing the diameter of the inhibition zone with information published by NCCLS (National Committee on Clinical Laboratory Standards) in Performance Standards for Antimicrobial Disk Susceptibility Testing, the subject matter of which is incorporated herein by reference. The results of this test are semi-quantitative in that there are three categories of susceptibility—namely resistant, intermediate and susceptible.

Another method of antimicrobial susceptibility testing is the antibiotic gradient method. This test utilizes an antibiotic gradient in a gel medium. Paper or plastic strips are impregnated with an antibiotic concentration gradient. A plurality of strips are placed on a Mueller-Hinton agar plate like spokes on a wheel, with the plate having been inoculated with the microorganism to be tested. After incubation, an antibiotic gradient is formed in the gel in an elliptical shape around each test strip (if the microorganism is susceptible to the antibiotic on the particular strip). The minimum concentration of the antimicrobial agent that prevents visible microorganism growth is the endpoint of the test (the minimum inhibitory concentration, or MIC). In other words, in disk diffusion testing, the MIC is the concentration at the edge of the inhibition zone (the growth/no growth boundary). In this case, the MIC is the point at which the elliptical growth inhibition area intersects the test strip.

A third type of susceptibility test is the broth microdilution test. In this type of test, dilutions of antibiotics (e.g. consecutive two-fold dilutions) are prepared. Often, at least ten concentrations of a drug are prepared in tubes or microwells. Each tube or well having the various concentrations of antibiotics is inoculated with a particular microorganism (a standardized suspension of test bacteria is added to each dilution to obtain a final concentration of 5×10⁵ CFU/ml). A growth control well and an uninoculated control well are included on each plate. After incubation (e.g. for 16-24 hours at 35 degrees C.), the wells or tubes are examined manually or by machine for turbidity, haze and/or pellet. Indicators can be placed in the wells to facilitate the visualization of microbial growth. As with other tests, the minimum concentration of antimicrobial agent that prevents visible microbial growth is the MIC.

Suitable candidate compounds can include antibiotics suitable for administering to patients in need of treatment for a bacterial infection, or any other suitable anti-microbial or therapeutic compound known to those skilled in the art, alone or in combination with each other including, but not limited to, beta-lactams (penicillin, ampicillin, piperacillin, imipenem), quinolones (levofloxacin, ciprofloxacin, norfloxacin, moxifloxacin), chloramphenicol, aminoglycosides (gentamicin, amikacin) glycopeptides (vancomycin, teioplanin), or antifungals (fluconazole, voriconazole, amphotericin B), or antibiotics combined with beta-lactamase inhibitors.

“Combination therapy” (or “co-therapy”) includes the administration of some anti-microbial compound contemplated by the present invention, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not intended to, encompass the administration of two or more of these therapeutic agents as part of separate monotherapy regimens that incidentally and arbitrarily result in the combinations contemplated by the present invention.

“Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies (e.g., surgery or radiation treatment.)

By exposing the at least one candidate compound to the resistant microorganism having a first and second molecular marker, quantifying the first and second markers, and determining the overall microbial mass to the viability of the microorganisms, candidate compounds can be effectively screened. A concordant result indicates that the candidate compound is not effective against the resistant microorganisms (because the microorganisms are still viable), whereas a discordant result indicates that the candidate compound is effective against the resistant microorganisms (because the microorganisms are no longer viable).

Following treatment of the microbial infection with a suitable antimicrobial, the methods of the invention can be used to monitor the treatment efficacy in the patient. Serial samples containing the microbial infection are obtained from the patient after administration of the antimicrobial and the first and second molecular markers for each sample of the microorganisms is quantified, e.g., by qPCR and RT-qPCR, respectively, and the ratio of overall microbial mass to microorganism viability is determined. The ratios are then compared at each time point. A concordant ratio over time indicates that the microorganisms have become resistant to the antimicrobial, while a discordant ratio over time indicates that the microorganisms are still susceptible to the antimicrobial.

The following EXAMPLES are presented in order to more fully illustrate the invention. These EXAMPLES should in no way be construed as limiting the scope of the invention, as defined by the appended claims.

### EXAMPLES

### Example 1

### Detection of the Presence and Viability of S. gordonni and Tol1 After Drug Exposure In Vitro by Real-Time qPCR and RT-qPCR

Technical Approach/Procedures:

1) Strains and growth conditions: Wild-type (wt) and penicillin tolerant (Tol) Streptococcus gordonii (I. Caldelari et al., Antimicrobial Agents and Chemotherapy, 2000, 44(10):2802-2810) were grown at 37 degrees Celsius, either in brain heart infusion broth (BHI) without aeration, or on Columbia agar supplemented with 3% blood. Quantitative culture was performed by plating serial dilutions of bacterial cultures taken at various time points on penicillinase-containing blood-agar plates. Colonies counts were determined after incubation for 48 hours days at 37° C.

2) Amplification of the 16S ribosomal gene: A 120 bp. fragment of the 16SrRNA-gene (Accession number D38483) was amplified by PCR using the following primer pair: (i) 5′-GGA AAC GAT AGC TAA TAC CGC ATAA-3′ (SEQ ID NO:1) and (ii) 5′-AAT CGA TCA TCC ACT CCA TTG CCG AG-3′ (SEQ ID NO:2). Reactions were carried on a 2400 GeneAmp PCR system (Perkin-Elmer) in a total volume of 50 μl 1×PCR-buffer (Gibco) containing 25 pmol of each primer and 2 UI of Taq DNA polymerase (Gibco). The following PCR conditions were applied for 25 cycles: (i) 94° C. during 30 seconds, (ii) 50° C. during 30 seconds and (iii) 72° C. during 20 seconds. Amplicons were isolated using the Quiaquick PCR-purification kit from Quiagen, prior ligation into the pGEM-T Easy vector system (Promega), and cloning into Escherichia coli. Plasmids were extracted using the Wizard Midiprep Kit (Promega). Concentration of extracted DNA was performed by spectrophotometry, and molar concentration determined using the following formula: 1 μg of a 1000 bp DNA fragment=1.52 pmol=1.52 10⁻¹² moles×N molecules, where N stands for the Avogadro number (6.023×10²³ molecules/mole). Different solutions were then accordingly prepared (10⁸-10² molecules/l) and used as standard solutions in the further experiments.

3) Extraction and purification of bacterial DNA and RNA: (i) Total DNA from 3 ml of culture samples was extracted and purified using the DNeasy Tissue Kit according to the instructions of the manufacturer (Qiagen). (ii) For total RNA isolation, 9 ml of culture samples were centrifuged at 10'000 rpm for 8 minutes at 4° C. and processed according to the FastRNA BLUE Protocol of BIO 101 modified as followed. Bacterial pellets were resuspended in 500 μl of CRSR-BLUE reagent and transferred into tubes containing glass beads, 500 μl of phenol acid and 100 μl a 24:1 chloroform-isoamylalcohol solution. Samples were further processed at 4° C. using a FastPrep apparatus (BIO 101, Savant) for 25 seconds at a speed of 6.5, before being centrifuged at 14'000 rpm for 10 minutes. The aqueous phase was collected and added to a 500 μl of the CIA solution. Samples were then centrifuged at 14'000 rpm for 5 minutes. The aqueous phase was collected and mixed with 350 μl of RLT Buffer (Qiagen) supplemented with 1% β₂-Mercaptoethanol and 250 μl RNase-free EtOH (96-100%). Total RNA was further purified according to the standard RNase-Free DNase Set Protocol of Qiagen. Total RNA concentrations were determined by spectrophotometry and quality checked on 1% MOPS-agarose gels.

16S rRNA-cDNA were synthesized from total RNA using the Omniscript RT-PCR Kit from Qiagen in a total volume-reaction of 20 μl as described.

4) Quantitative Real-Time PCR was performed using the following probe FAM-5′-TTG CAC CAC TAC CAG ATG GAC CTGC-3′-TAMRA (SEQ ID NO:3) according to the instruction of the manufacturer (Perkin-Elmer) on Sequence Detection System 5700 (Perkin-Elmer) in a total volume of 50 μl 1×PCR buffer (Gibco) containing 25 pmoles of each primer, 120 nmoles of fluorescent probe, 300 μmoles each dNTP and 1.5 UI of Taq DNA polymerase (Gibco). The following program was applied during 40 cycles: (i) 94° C. for 15 sec., (ii) 52° C. for 30 sec., (iii) and 72° C. for 30 sec. Each experiment was done in triplicate. DNA content of each sample was determined using a standard curve obtained by processing in the same time samples containing fixed DNA concentrations (see above section).

Results:

1) Qualitative PCR: Qualitative PCR gave the same results for ribosomal DNA vs. ribosomal RNA. Qualitative PCR of chromosomal genes was highly positive during the whole growth curve and remained positive whether the cultures were viable or had been dramatically killed with penicillin, e.g., loss of viability >3 log CFU/ml. Reverse transcription of 16S ribosomal RNA stretches followed by qualitative PCR gave the same results. Therefore, the results prohibited any correlation between PCR amplification and bacterial viability.

1) Identification of a stable marker: Quantitative real time PCR of DNA from chromosomal 16S-rRNA gene was highly positive during the whole bacteria growth curve and correlated with the number of bacteria recovered from colony counts, as determined on agar plates. Thus quantitative determination of 16S-rRNA DNA was proportional to bacterial viable titers during growth in absence of antibiotic. After addition of penicillin however, the PCR products stopped increasing and remained constant over time and stable for up to 50 hours, despite a loss of viability of 5 log CFU/ml during the same period of time. Thus, real-time PCR of DNA stretches was a stable marker (for >50 hours) of the presence of bacterial bodies, but not of bacterial viability, i.e., antibiotic-induced death. This was as predicted if DNA were to be a stable marker persisting for a prolonged period of time after cell death

2) Identification of a marker correlating with cell viability: Quantitative real time PCR determination of 16S rRNA was performed after reverse transcription of 16S rRNA from total RNA and characterized amplicons that decreased proportionally to cell death during penicillin-induced killing. The results were very reproducible in independent experiments. This was as predicted if RNA were to be an unstable marker, that correlated with viability. Moreover, when the same system was tested with the penicillin-tolerant mutant Tol as control, the ribosomal RNA amplicons remained stable and proportional to viable bacteria as expected.

The above described system was also reproducible with other antibiotics such as levofloxacin, the DNA-gyrase inhibitor. As with penicillin-induced killing, levofloxacin-induced killing was accompanied by the persistence of the chromosomal DNA amplicons acting as a bacterial mass marker, and a decrease in the specific ribosomal RNA amplicon acting as a marker of cell viability. Thus, the ribosomal DNA/ribosomal RNA ratio provided information on the ratio between the overall bacterial mass and the bacteria that were still viable.

### Other Embodiments

From the foregoing detailed description of the specific embodiments of the invention, it should be apparent that unique methods of assessing the presence and viability of microorganisms, such as bacteria, have been described. Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims. For instance, the choice of the particular system or pathogen for which the methods can be performed in is believed to be a matter of routine for a person of ordinary skill in the art with knowledge of the embodiments described herein.

What is claimed is:

1. A method for assessing viability of a microorganism comprising:
a) administering an antimicrobial to a microorganism population having a first marker and a second marker;
b) quantifying said first marker following administration of the antimicrobial;
c) quantifying said second marker following administration of the antimicrobial; and
d) determining the ratio between the quantity of the first marker and the quantity of the second marker; and
wherein a positive-positive ratio indicates that microorganisms are present and viable, and wherein a positive-negative ratio indicates that microorganisms are present but non-viable.

2. The method of claim 1, wherein said first marker comprises a stable molecule of the microorganism.

3. The method of claim 2, wherein said stable molecule comprises a chromosomal DNA molecule or a plasmid DNA molecule.

4. The method of claim 3, wherein the chromosomal DNA molecule is 16S rRNA DNA.

5. The method of claim 2, wherein said first marker persists for a prolonged period of time following administration of the antimicrobial.

6. The method of claim 5, wherein the prolonged period of time is from about 1 minute to about 50 hours.

7. The method of claim 1, wherein said second marker comprises an unstable molecule of the microorganism.

8. The method of claim 7, wherein said unstable molecule comprises an mRNA molecule or a ribosomal RNA molecule.

9. The method of claim 8, wherein the ribosomal RNA molecule is 16S ribosomal RNA.

10. The method of claim 1, wherein the quantifying of step (b) is performed using Real-Time Quantitative PCR.

11. The method of claim 1, wherein the quantifying of step (c) is performed using reverse transcription followed by Real-Time Quantitative PCR.

12. The method of claim 1, wherein said antimicrobial is selected from the group consisting of: beta-lactams; penicillin; ampicillin; piperacillin; imipenem; quinolones; levofloxacin; ciprofloxacin; norfloxacin; moxifloxacin; chloramphenicol; aminoglycosides; gentamicin; amikacin; glycopeptides; vancomycin; teicoplanin; antifungals; fluconazole; voriconazole; and amphotericin B.

13. A method of detecting the presence or absence of a microorganism in a test sample of interest, the method comprising:
a) determining the presence or absence of a stable marker that persists for a prolonged period of time in said test sample following treatment with an antimicrobial; and
c) quantifying said stable marker, if present,
wherein the presence of the stable marker following administration of the antimicrobial indicates the presence of the microorganism in the test sample, and wherein the absence of the stable marker indicates the absence of the microorganism in the test sample.

14. The method of claim 13, wherein said stable marker comprises a stable molecule of the microorganism.

15. The method of claim 14, wherein said stable molecule comprises a chromosomal DNA molecule or a plasmid DNA molecule.

16. The method of claim 13, wherein said test sample comprises a member selected from the group consisting of: any mammalian tissue; any mammalian secretion; a specimen derived from a food; a specimen derived from a drinking supply; a specimen suspected of being related to a biological weapon; and a potential biological weapon.

17. The method of claim 16, wherein said mammalian tissue or secretion is human.

18. The method of claim 16, wherein said specimen suspected of being related to a biological weapon is a biological storage container.

19. The method of claim 13, wherein said test sample is provided in vitro.

20. A method of determining the efficacy of a treatment for an infection by a microorganism having a first and a second marker comprising:
a) administering the treatment to a subject having the infection that requires such treatment;
b) obtaining a sample from said subject;
c) quantifying a first marker in said sample following administration of the treatment;
d) quantifying a second marker in said sample following administration of the treatment; and
e) determining the ratio between the quantity of the first marker and the quantity of the second marker,
wherein a positive-positive ratio indicates that the microorganism is present and viable following administration of the treatment and that the treatment lacks efficacy, and wherein a positive-negative ratio indicates that the microorganism is present but is now non-viable following administration of the treatment and that the treatment is efficacious.

21. The method of claim 20, wherein said first marker comprises a stable molecule of the microorganism.

22. The method of claim 21, wherein said stable molecule comprises a chromosomal DNA molecule or a plasmid DNA molecule.

23. The method of claim 22, wherein said chromosomal DNA molecule is 16S rRNA DNA.

24. The method of claim 20, wherein said first marker persists for a prolonged period of time following administration of the treatment.

25. The method of claim 24, wherein the prolonged period of time is from about 1 minute to about 50 hours.

26. The method of claim 20, wherein said second marker comprises an unstable molecule of the microorganism.

27. The method of claim 26, wherein said unstable molecule comprises an mRNA molecule or a ribosomal RNA molecule.

28. The method of claim 27, wherein the ribosomal RNA molecule is 16S ribosomal RNA.

29. The method of claim 20, wherein the quantifying of step (c) is performed using Real-Time Quantitative PCR.

30. The method of claim 20, wherein the quantifying of step (d) is performed using reverse transcription followed by Real-Time Quantitative PCR.

31. The method of claim 20, wherein the treatment comprises an antimicrobial.

32. The method of claim 31, wherein said antimicrobial is selected from the group consisting of: beta-lactams; penicillin; ampicillin; piperacillin; imipenem; quinolones; levofloxacin; ciprofloxacin; norfloxacin; moxifloxacin; chloramphenicol; aminoglycosides; gentamicin; amikacin; glycopeptides; vancomycin; teicoplanin; antifungals; fluconazole; voriconazole; and amphotericin B.

33. The method of claim 20, wherein said subject is a mammal.

34. The method of claim 33, wherein said mammal is a human.

35. The method of claim 20, further comprising repeating steps (a)-(e) in order to monitor the efficacy of the treatment over time.

36. A method of assessing antimicrobial tolerance, resistance, or susceptibility of a microorganism, the method comprising:
a) administering an antimicrobial to the microorganism having a first marker and a second marker;
b) quantifying said first marker following administration of the antimicrobial;
c) quantifying said second marker following administration of the antimicrobial; and
d) determining the ratio between the quantity of the first marker and the quantity of the second marker,
wherein a positive-positive ratio indicates that the microorganism is resistant or tolerant to the antimicrobial, and wherein a positive-negative ratio indicates that the microorganism is susceptible to the antimicrobial.

37. A method for diagnosing a microbial infection in a patient, the method comprising
a) obtaining at least one sample from the patient; and
b) detecting the presence or absence of a microorganism in the sample using the method of claim 13,
wherein the presence of microorganisms in the sample is indicative of a microbial infection.

38. A method of selecting a treatment for a patient with a microbial infection, the method comprising:
a) obtaining at least one sample from the patient, said sample containing a microorganism having a first marker and a second marker;
b) administering an antimicrobial to the sample;
c) quantifying said first marker following administration of the antimicrobial;
d) quantifying said second marker following administration of the antimicrobial;
e) determining the ratio between the quantity of the first marker and the quantity of the second marker, wherein a positive-positive ratio indicates that the microorganisms are resistant or tolerant to the antimicrobial, and wherein a positive-negative ratio indicates that the microorganisms are susceptible to the antimicrobial;
f) selecting the antimicrobial for continued administration to the patient, provided that the ratio between the first marker and the second marker is positive-negative, or repeating steps (b)-(f) with an alternative antimicrobial if the ratio between the first marker and the second marker for the first antimicrobial is positive-positive.

39. A method of monitoring treatment efficacy in a patient having a microbial infection, the method comprising:
a) obtaining serial samples from a patient undergoing treatment for a microbial infection; and
b) repeating the steps of the method of claim 1 on each of said samples; and
c) comparing the ratios determined at each time point,
wherein the development of a positive-positive ratio over time indicates that the microorganisms have become resistant or tolerant to the antimicrobial, and wherein a positive-negative ratio over time indicates that the microorganisms have remained susceptible to the antimicrobial.

40. A method of screening at least one candidate compound for efficacy against resistant or tolerant microorganisms, the method comprising:
a) exposing the at least one candidate compound to the resistant or tolerant microorganism, said microorganism having a first marker and a second marker;
b) quantifying said first marker following the exposure step;
c) quantifying said second marker following the exposure step; and
d) determining the ratio between the quantity of the first marker and the quantity of the second marker,
wherein a positive-positive ratio indicates that the at least one candidate compound is not effective against said resistant or tolerant microorganism, and wherein a positive-negative ratio indicates that the at least one candidate compound is effective against said resistant or tolerant microorganism.

41. The method of claim 40, wherein the at least one candidate compound is selected from the group consisting of: beta-lactams; penicillin; ampicillin; piperacillin; imipenem; quinolones; levofloxacin; ciprofloxacin; norfloxacin; moxifloxacin; chloramphenicol; aminoglycosides; gentamicin; amikacin; glycopeptides; vancomycin; teicoplanin; antifungals; fluconazole; voriconazole; and amphotericin B.

42. The method of claim 1, wherein the microorganism is a member selected from the group consisting of: prokaryotes; fungi; viruses; and parasites.

43. The method of claim 42, wherein the prokaryote is a bacterium.

44. The method of claim 43, wherein the bacterium is S. gordonni.


SEQUENCE LISTING (3 sequences)

SEQ ID NO: 1 - DNA, 25 bp, artificial
  PCR primer/adaptor
ggaaacgatagctaataccgcataa

SEQ ID NO: 2 - DNA, 26 bp, artificial
  PCR primer/adaptor
aatcgatcatccactccattgccgag

SEQ ID NO: 3 - DNA, 25 bp, artificial
  PCR primer/adaptor
ttgcaccactaccagatggacctgc